(19) United States
(12) Patent Application Publication
(10) Pub. No.: US 2005/0071012 A1
(43) Pub. Date: Mar. 31, 2005

(54) Methods and devices to replace spinal disc nucleus pulposus

(76) Inventors: Hassan Serhan, South Easton, MA (US); Michael Slivka, Taunton, MA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/676,869

(22) Filed: Sep. 30, 2003

(51) Int. Cl. A61F 2/44

(52) U.S. Cl. 623/17.16

(57) ABSTRACT

A minimally invasive nucleus pulposus augmentation or replacement methods and devices are disclosed. The method relates to insertion of nucleus pulposus augmentation or replacement materials help maintain disc height and promote regeneration of the native nucleus pulposus structure.

### BACKGROUND OF THE INVENTION

1. Field of the Invention

This invention is concerned with methods and devices for treatment for back pain caused by defects in the intervertebral disc by repairing and/or restoring the nucleus pulposus.

2. Related Art

Injury and/or degeneration of the intervertebral disc can cause back pain as a result of disc herniation, rupture of the annulus and/or prolapse of the nucleus pulposus. Herniation and nucleus prolapse can cause spinal canal and foraminal stenosis. All may cause release of chemotactic factors that irritate the spinal cord. Acute damage to the annulus and/or nucleus prolapse can cause abnormal biomechanical function of the disc and subsequent disc degeneration.

Discectomy, laminectomy, laminotomy and/or spine fusion procedures represent state of the art surgical treatment for disc problems. Heating the disc using a probe has been suggested to “weld” defects. Injecting curable materials into the nucleus has also been suggested to act as filler material for the nucleus and annular defect.

A few disc prosthesis devices and nucleus pulposus augmentation devices are being investigated on a limited basis. The nucleus pulposus augmentation devices being evaluated are either in situ cured (in-situ cured polyurethane contained in a bag and in-situ cured protein polymers) or relatively solid hydro-gels (Ray Medical hydro-gel in UHMWPe pillow and Howmedica hydro-gel ball). In situ cured nucleus pulposus augmentation injectable augmentation devices has the potential to ooze and seeps out of the disc space intra-operatively.

Lambrecht et. al (PCT/WO0112107A1) disclose a barrier prosthesis such as a plug made of biocompatible material with anchoring means for repairing the annulus and supporting the nucleus pulposus. Disclosed materials include flexible, biocompatible materials, fibrous materials such as collagen or cellulose, and hydrogels. Also disclosed are porous materials that provide tissue ingrowth and bioabsorbable materials, although these are not presented as preferred embodiments.

Ferree (PCT/WO0110316, U.S. Pat. No. 6,245,107) discloses treatment of annular defects using a material which is inserted into the disc in a first insertable state and then is allowed to expand, return or solidify into a second state which occludes the defect. Bioabsorbable materials are mentioned but no disclosure is made regarding materials that are tissue conductive, and no mention is made of SIS. Haldimann (PCT/WO0062832) discloses an in-situ curable polymeric adhesive that is used to fill the disc defect and adhere to the adjacent tissues. Guagliano and Ross (U.S. Pat. No. 6,206,921 B1) disclose a similar system to Haldimann where an injectable, setting, resilient material is used to replace the nucleus pulposus. Stovall (PCT/WO9904720) discloses using a cell containing hydrogel to treat herniated discs. Bao and Yuan (PCT/WO9961084) disclose an expandable, porous material to seal biological apertures and permit tissue ingrowth. Felt et al. (U.S. Pat. No. 6,140,452) disclose an injectable, curable polyurethane to repair tissue sites. Sharkey et al. (U.S. Pat. No. 6,126,682) disclose a method of heating the annulus to weld the defect that can be coupled with a delivery of sealing agents. Gan et al. (U.S. Pat. No. 5,964,807) disclose porous hybrid materials containing sol gel bioactive material that can be used to repair the disc. Plouhar et al. (U.S. Pat. No. 5,922,028) disclose a tissue graft consisting of secured layers of intestinal submucosa which is sculptured to have the anatomical shape of the cartilaginous structure that is to be repaired.

However, the prior art does not disclose any devices or methods whereby a degenerated nucleus pulposus is replaced with biocompatible, implants modeled preferably on collagen scaffolds and preferably derived from porcine small-intestinal submucosa (SIS). Such devices are envisioned as being capable of being adequate devices for restoring disc height and maintaining adequate disc motion as hereinafter described and claimed.

### BRIEF DESCRIPTION OF THE DRAWINGS

FIG. 1a depicts a longitudinal view of a segment of small intestine submucosa (SIS) material with a perforation representing an incision line.

FIG. 1b shows the SIS material of FIG. 1a after having been outstretched and at the start of rolling the material.

FIG. 1c depicts the fully rolled material of FIG. 1b with perforations indicating where the rolled material is to be cut.

FIG. 1d shows the rolled segments of the rolled material of FIG. 1c after cutting at the perforations of FIG. 1c.

FIG. 2 shows a minimally invasive procedure of inserting materials into a spinal disc to augment or replace the nucleus pulposus.

FIG. 3 is a representation of a suitable insertion tool for the segments of material to be inserted into the nucleus pulposus region of a spinal disc.

### SUMMARY OF THE INVENTION

One embodiment if this invention relates to a minimally invasive method of augmenting or replacing of nucleus pulposus of a spinal disc comprising the steps of:
a) preparing a disc treatment site;
b) piercing and inserting into and through the sidewall of the disc's annular ring a cannulated insertion tool; and
c) inserting small intestine submucosa (SIS) through the cannulated insertion tool and into the nucleus pulposus.

Another embodiment of this invention relates to a minimally invasive method of augmenting or replacing of nucleus pulposus of a spinal disc comprising the steps of:
a) preparing a disc treatment site;
b) piercing and inserting into and through the sidewall of the disc's annular ring a cannulated insertion tool; and
c) inserting an elongated nucleus pulposus augmentation or replacement material through the cannulated insertion tool and into the nucleus pulposus.

Preferred forms of the SIS and nucleus pulposus augmentation or replacement materials are elongate and may take the form strips, cords, braids, tubes, rolls and pellets and combinations thereof.

As hereinafter disclosed and claimed further embodiments of this invention include providing and using the SIS and nucleus pulposus augmentation or replacement materials that has been seeded with cells and/or treated with bioactive factors.

Advantages of the invention include the fact that it provides minimally invasive approach to disc repair particularly in, maintaining disc height, resisting nucleus leakage and in preferred embodiments promoting regeneration of the native nucleus pulposus structure.

### DETAILED DESCRIPTION OF PREFERRED EMBODIMENTS OF THE INVENTION

One embodiment of this invention relates to a device and method for augmenting or replacing the nucleus pulposus of a spinal disc with small intestinal submucosa (SIS).

SIS is a naturally occurring extracellular collagen based matrix. SIS is described in detail in U.S. Pat. No. 5,372,821, the disclosure of which is hereby incorporated by reference. As described in the '821 patent, SIS is a segment of intestinal tissue of a warm-blooded vertebrate, said segment comprising the tunica submucosa and basilar tissue of the tunica mucosa, said tunica submucosa and basilar tissue being delaminated from the tunica muscularis and the luminal portion of the tunica mucosa of said segment of intestinal tissue. SIS contains cytokines and growth factors and has been shown to act as a resorbable scaffold in vivo which promotes soft tissue regeneration with little scar tissue formation. SIS can be manufactured in laminated sheets of various sizes and thicknesses for different indications. Successful_applications of SIS have included: dural substitution, rotator cuff repair, tendinosis, vessel repair, abdominal and bladder wall repair, and others. However, prior to investigations initiated and directed by the inventors, SIS is not known to have been investigated to determine its ability to facilitate regeneration disc defects.

SIS used with this invention is desirably delivered to the nucleus pulposus part of a spinal disc in a minimally invasive fashion. To this end, the geometry of the SIS may be tailored to accomplish goal.

In some embodiments, the nucleus pulposus implant comprises an elongate form such that the narrow dimension allows the material to be inserted through a cannula and through the defect, incision or hole created in the annulus. A microdiscectomy is sometimes carried out through a 5 mm trephine hole created in the annulus. An example of an appropriate elongate material for insertion through a 5 mm hole would be a pellet having a diameter of 5 mm and length of 10 mm.

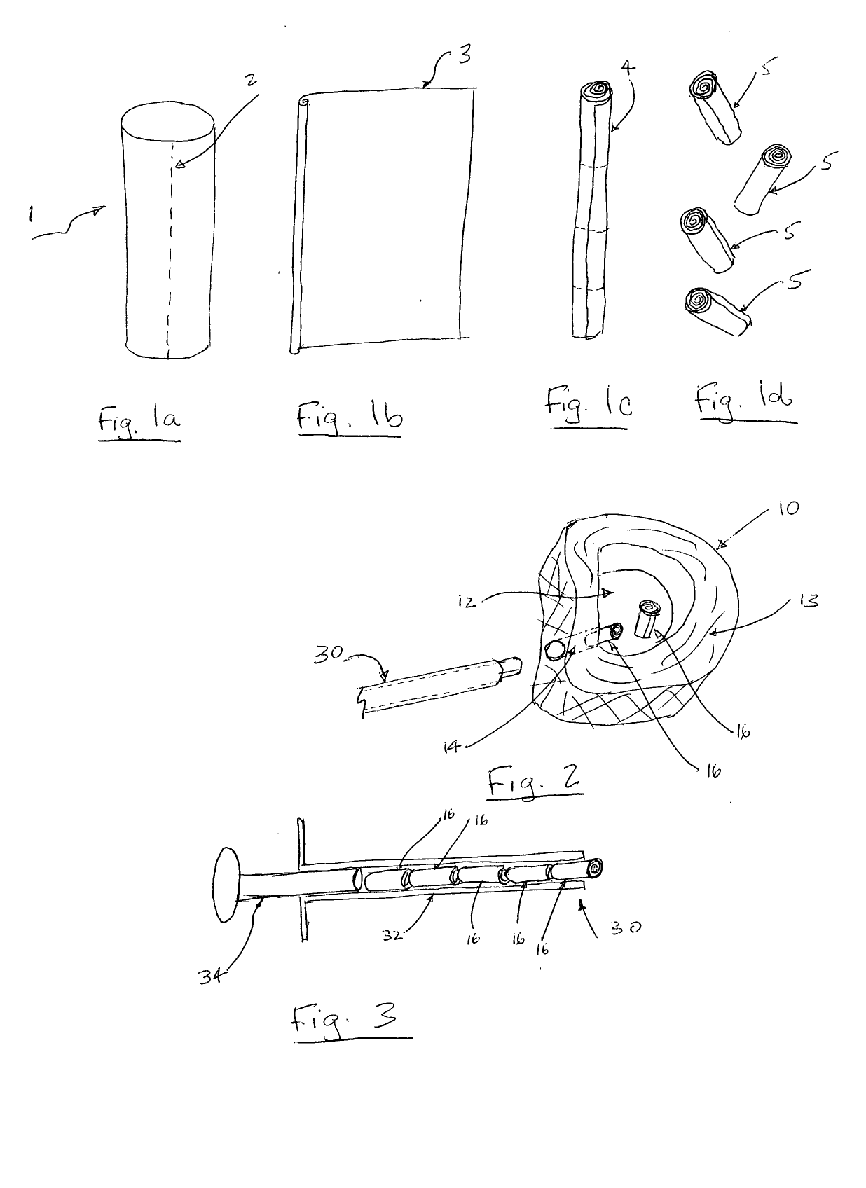

FIG. 1a to 1d depict preparation of a preferred pellet form of the SIS in elongate form. Referring to FIG. 1a, naturally occurring SIS 1 is cut along perforated line 2 and extended or stretched to form a sheet (not shown). FIG. 1b depicts the SIS in the form of a sheet 3 which has begun to be rolled upon itself to the appropriate diameter to form a SIS roll 4 as shown in FIG. 1c. Optionally, SIS roll 4 is cut into discrete lengths 5 such that no intraoperative cutting to length is required as shown in FIG. 1d.

Thus one embodiment of this invention relates to a method of preparing small intestine submucosa (SIS) implant comprising the steps of:
a) providing a source of SIS;
b) cutting open the SIS to form a sheet; and
c) rolling the SIS sheet to a desired diameter.

Alternatively, a cruciate incision may be made in the annulus. The elongate dimension is sufficiently large to mitigate extrusion of the material out of the nucleus pulposus at any orientation different from that in which it was inserted. Suitable forms include strips, cords, braids, tubes, rolls and pellets, for example. Packing the disc with these materials allows for efficient filling while mitigating extrusion, and further provides structural support to prevent disc space narrowing.

In other embodiments, the nucleus pulposus implantation comprises injecting a comminuted form or a multitude of particulates. These forms have the advantage of having a high surface area for tissue ingrowth. Some examples of suitable comminuted or particulate materials include fibers, powder, spheres, and granules. The particulates may be suspended in any biocompatible media to facilitate delivery of the material and may contain agents to promote tissue ingrowth and cell differentiation (list).

In yet other embodiments the particulates may be combined with the elongate forms mentioned previously to combine the advantages of the two approaches.

In yet a further embodiment, the particulate form is combined with the elongate form to create a composite pre-formed structure. For example, comminuted SIS in the form of fibers may be rolled into a sheet of SIS (as described earlier), then optionally cut to form composite pellets.

Other embodiments of the invention contemplate augmenting the nucleus implants with a lubricating medium to ease insertion of the materials into the disc space and in some instances provide cells to aid in new tissue growth in the augmented repair area. Examples include hyaluronic acid, platelet-rich plasma and bone marrow aspirate.

In another embodiment of the invention, the nucleus pulposus augmentation or replacement material is comprised of a biocompatible porous material, i.e., a material that is not harmful to and does not cause an undesirable immunological response in a body, e.g., a human being. The biocompatible material may be non-bioabsorbable or bioabsorbable.

As with the SIS material, the porous nature of the nucleus pulposus augmentation or replacement material allows for the material to act as a scaffold for cells to occupy and produce extracellular matrix. Repair cells may migrate from the surroundings following implantation or be seeded onto the repair material prior to implantation. Additionally, bioactive factors may be applied to or incorporated into the nucleus pulposus augmentation or replacement material and SIS material.

Examples of non-bioabsorbable nucleus pulposus augmentation or replacement materials include, but are not limited to polyacrylates, ethylene-vinyl acetates (and other acyl-substituted cellulose acetates), polyester (Dacron®), poly(ethylene terephthalate), polypropylene, polyethylene, polyurethanes, polystyrenes, polyvinyl oxides, polyvinyl fluorides, poly(vinyl imidazoles), chlorosulphonated polyolefins, polyethylene oxides, polyvinyl alcohols (PVA), polytetrafluoroethylenes, nylons, and combinations thereof.

The nucleus pulposus augmentation or replacement materials of this invention is preferably a porous, bioabsorbable material that is tissue conductive and is desirably, eventually completely replaced by repair tissue. Thus the disc defect repair acts as a temporary support structure for tissue regeneration and resulting in a primarily native repair tissue structure. Preferably the breakdown products of the invention are easily processed by the body through normal metabolic pathways.

Suitable bioabsorbable nucleus pulposus augmentation or replacement materials include collagen, hyaluronic acid, elastin, albumin, reticulin, prolamines, polysaccharides, alginate, heparin, biodegradable polymers of sugar units, synthetic polymers including polylactide, polyglycolide, polydioxanone, polyhydroxybutyrate, polyhydroxyvalerate, poly(propylene fumarate), polyoxaesters, synthetic polyamino acids, biodegradable polyurethanes and their copolymers, and combinations thereof. In one preferred embodiment of this invention, the porous repair material is a textile structure comprised of drawn fibers of the aforementioned materials. In a more preferred embodiment, the fibers are woven or braided into the appropriate scaffold structure mentioned.

The method of this invention may be more fully understood by reference to the FIGS. 2 and 3.

FIG. 2 depicts a cross-sectional view of disc 10 comprising nucleus pulposus area 12, annular fibrosus or annular ring 13. Through the sidewall of annular ring 13 is inserted a cannula to provide pathway 14 for the nucleus pulposus augmentation or replacement material 16 to be inserted. FIG. 2 actually depicts some material 16 in pathway 14 and some within the nucleus pulposus area 12 of disc 10. A cannulated delivery tool 30 is used to deliver material 16 into the nucleus pulposus.

FIG. 3 represents a tool 30 suitable for delivery of material 16 into the nucleus pulposus 12. Specifically tool 20 comprises a cannulated delivery tube 32 and plunger 34. In the depicted embodiment, nucleus pulposus augmentation or replacement material 16 is represented by segments. However, it is understood that segments may be replaced or used in addition to other types or forms of nucleus pulposus augmentation or replacement material 16 such as the commutated forms and particulate forms described above.

Thus, the minimally invasive method of this invention in its essential form comprises the steps of:
a) preparing a disc treatment site;
b) piercing and inserting into and through the sidewall of the disc's annular ring a cannulated insertion tool; and
c) inserting small intestine submucosa (SIS) through the cannulated insertion tool and into the nucleus pulposus.

Additionally, another embodiment is related to a minimally invasive method comprising the steps:
a) preparing a disc treatment site;
b) piercing and inserting into and through the sidewall of the disc's annular ring a cannulated insertion tool; and
c) inserting an elongated nucleus pulposus augmentation or replacement material through the cannulated insertion tool and into the nucleus pulposus.

Alternately, the two foregoing methods may be modified in such a way that an insertion is made in the annulus and the cannula is placed in proximity of the insertion (i.e., not through the insertion) and the SIS or elongated nucleus pulposus augmentation or replacement material is introduced through the annular hole and into the nucleus pulposus.

The method also contemplates the step of suturing the pathway created by the cannulated delivery tool after delivery of the nucleus pulposus augmentation or replacement material and removal of the delivery tool. The suturing should easily be accomplished due to the elastic nature of the annular which should return to nearly the same state it was prior to the annular ring being pierced by the cannulated delivery tool.

Insertion is possible due to the elastic nature of the annulus. The diameter of the tail region is preferably the same diameter or slightly larger than the annular defect to ensure complete filling.

In some embodiments, the above materials are augmented with an adhesive or sealant material to aid in sealing of the annular ring insertion hole formed by the cannulated tool to prevent herniation around the insertion hole following implantation. Potential materials include platelet-rich plasma clotted with thrombin, fibrin glue, cyanoacrylates, crosslinked proteins (such as gluteraldehyde and albumin) and polymers, and muscle adhesive protein.

The invention also contemplates that the SIS material or the nucleus pulposus augmentation or replacement material of this invention may be contacted or otherwise cultured with tissue repair cells for a period of time prior to implantation. Alternatively, bioactive factors may be adsorbed onto or absorbed into the repair material prior to implantation.

Examples of suitable repair cells include cells harvested from spinal discs in the body such as nucleus pulposus cells and annulus fibrosis cells. Other examples include but are not limited to: stem cells, bone marrow cells, fibrocytes, adipocytes and chondrocytes.

Additionally, suitable repair cells may be derived from soaking, coating, or otherwise contacting the SIS or nucleus pulposus augmentation or replacement material in bone marrow aspirate, platelet rich plasma, platelet poor plasma, whole blood, serum or other autologous media.

Examples of suitable bioactive factors include but are not limited to transforming growth factor-beta and agents in the same family of growth factors, platelet-derived growth factors, fibroblast growth factors, insulin-like growth factors, protein polymers such as RGD-peptides and Indian Hedgehog proteins, anti-inflammatory agents, angiogenic factors, hormones, hyaluronic acid and the like.

More specific examples of suitable transforming growth factor-beta and agents in the same family of growth factors, include, but are not limited to, TGF-β1, TGF-β2, and TGF-β3, GDF-5, MP52, and BMPs (bone morphogenetic proteins).

Additionally, the vertebral endplates may be decorticated “curretted/picked” to cause bleeding into the disc space to allow adequate nutritional supply for the SIS or nucleus pulposus augmentation or replacement material remodeling.

It should be understood that the foregoing disclosure and description of the present invention are illustrative and explanatory thereof and various changes in the size, shape and materials as well as in the description of the preferred embodiment may be made without departing from the spirit of the invention.

1) A minimally invasive method of augmenting or replacing of nucleus pulposus of a spinal disc comprising the steps of:
a) preparing a disc treatment site;
b) piercing and inserting into and through the sidewall of the disc's annular ring a cannulated insertion tool; and
c) inserting small intestine submucosa (SIS) through the cannulated insertion tool and into the nucleus pulposus.

2) The method of claim 1, wherein the SIS is in an elongated form.

3) The method of claim 2, wherein the elongated form is selected from the group consisting of strips, cords, braids, tubes, rolls and pellets and combinations thereof.

4) The method of claim 4, wherein the elongated form is a pellet.

5) A minimally invasive method of augmenting or replacing of nucleus pulposus of a spinal disc comprising the steps of:
a) preparing a disc treatment site;
b) piercing and inserting into and through the sidewall of the disc's annular ring a cannulated insertion tool; and
c) inserting an elongated nucleus pulposus augmentation or replacement material through the cannulated insertion tool and into the nucleus pulposus.

6) The method of claim 5, wherein the form of elongated material is selected from the group consisting of strips, cords, braids, tubes, rolls and pellets and combinations thereof.

7) The method of claim 6, wherein the elongated form is a pellet.

8) A method of preparing small intestine submucosa (SIS) implant comprising the steps of:
a) providing a source of SIS;
b) cutting open the SIS to form a sheet; and
c) rolling the SIS sheet to a desired diameter.

9) The method of claim 8, further comprising the step of cutting the rolled sheet of SIS.

10) The method of claim 9, further providing particulate or commutated forms of SIS to be included during the rolling step of forming the SIS sheet.

11) The methods of claims 1-10, further comprising the presence of a bioactive factor or seeding cells in the SIS or nucleus pulposus augmentation or replacement material.

12) The method of claim 11, wherein the bioactive factor is selected group the group consisting of transforming growth factor-beta and agents in the same family of growth factors, platelet-derived growth factors, fibroblast growth factors, insulin-like growth factors, protein polymers such as RGD-peptides and Indian Hedgehog proteins, anti-inflammatory agents, angiogenic factors, hormones, hyaluronic acid and combinations thereof.

13) The method of claim 12, wherein the transforming growth factor-beta and agents in the same family of growth factors, are not limited TGF-β1, TGF-β2, and TGF-β3, GDF-5, MP52, and BMPs (bone morphogenetic proteins).

14) The method of claim 11, wherein the seeding cells are selected from the group consisting of stem cells, bone marrow cells, fibrocytes, adipocytes, chondrocytes, cells harvested from spinal discs in the body such as nucleus pulposus cells and annulus fibrosis, and combinations thereof.

15) The method of claim 14, wherein the seeding cells are stem cells.
